(19) United States
(12) Patent Application Publication
(10) Pub. No.: US 2022/0111002 A1
(43) Pub. Date: Apr. 14, 2022

(54) TREATMENT OF SENSORINEURAL DEAFNESS

(71) Applicant: UNIVERSITY OF MIAMI, Miami, FL (US)

(72) Inventor: Mustafa Tekin, Coral Gables, FL (US)

(21) Appl. No.: 17/612,870

(22) PCT Filed: May 21, 2020
(86) PCT No.: PCT/US20/34066

§ 371 Date: Nov. 19, 2021

Related U.S. Application Data
(60) Provisional application No. 62/850,935, filed on May 21, 2019.

(51) Int. Cl. A61K 38/17; A61K 31/7088; A61K 31/7105; A61K 38/46; A61P 27/16

(52) U.S. Cl. CPC A61K 38/177; A61K 31/7088; A61P 27/16; A61K 38/465; A61K 31/7105

(57) ABSTRACT

The present disclosure relates to a method of treating sensorineural deafness. The disclosure provides a method of treating sensorineural deafness in a mammalian subject (e.g., human) in need thereof. The method comprises administering to a subject having a mutation in a CLDN9 gene a composition that comprises a polynucleotide that encodes a CLDN9 peptide, a CLDN9 peptide, an agent that blocks expression of a mutant CLDN9 gene, an agent that corrects the mutation in the CLDN9 gene.

### CROSS-REFERENCE TO RELATED APPLICATION

This application claims the benefit of priority of U.S. Provisional Patent Application No. 62/850,935, filed May 21, 2019, which is hereby incorporated by reference in its entirety.

### GRANT FUNDING DISCLOSURE

This invention was made with government support under grant number DC009645 awarded by the National Institutes of Health (NIH). The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 54334_Seqlisting.txt; Size: 5,303 bytes; Created: May 21, 2020.

### FIELD OF THE INVENTION

The present disclosure relates to methods of characterizing and treating sensorineural deafness.

### BACKGROUND

Hearing loss (HL) is the most common sensory deficit, affecting approximately 1 in 650 newborns (Mehl and Thomson 2002). Genetic factors account for at least half of congenital and prelingual-onset HL (Morton and Nance 2006). Hearing loss may be classified as syndromic, which presents additional clinical features, or nonsyndromic, in which there are no other clinical findings. Nonsyndromic HL comprises about 70-80% of genetic deafness (Nance 2003). To date, 115 nonsyndromic HL genes have been identified. However, comprehensive genetic screening that includes all known deafness genes still leaves over 50% of cases unsolved (Shearer and Smith 2015).

### SUMMARY

The disclosure provides a method of treating sensorineural deafness in a mammalian subject (e.g., human) in need thereof. The method comprises administering to a subject having a mutation in a CLDN9 gene a composition that comprises a polynucleotide that encodes a CLDN9 peptide, a CLDN9 peptide, an agent that blocks expression of a mutant CLDN9 gene, an agent that corrects the mutation in the CLDN9 gene. Administration of a combination of any of the foregoing is also contemplated. In various aspects, the method comprises detecting the presence of a mutation in the CLDN9 gene in a sample from a subject. In various embodiments, the CLDN9 mutation is a DNA variant classified as pathogenic or likely pathogenic according to American College of Medical Genetics and Genomics (ACMG) criteria. The disclosure also contemplates a method comprising diagnosing the subject with sensorineural deafness when the presence of a mutation in the CLDN9 gene is detected.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment,” “some embodiments,” “various embodiments,” “related embodiments,” each of these types of embodiments is a non-limiting example of a feature that is intended to be combined with any other feature, or combination of features, described herein without having to list every possible combination. Such features or combinations of features apply to any of the aspects of the invention.

The headings herein are for the convenience of the reader and not intended to be limiting. Additional aspects, embodiments, and variations of the invention will be apparent from the Detailed Description and/or drawings and/or claims.

### BRIEF DESCRIPTION OF THE DRAWINGS

FIGS. 1A-1D. Characteristics of the family and the CLDN9 mutation. FIG. 1A. Turkish family with congenital hearing loss (black symbols) and genotypes at CLDN9 c.86delT. Double lines indicate consanguinity. FIG. 1B. Hearing thresholds obtained from pure-tone audiograms of the family. Affected individuals show moderate to profound hearing loss. FIG. 1C. Electropherograms showing the CLDN9 variant. WT: wild type, Horn: homozygous mutant, Het: heterozygous mutant. FIG. 1D. The frameshift occurs in extracellular loop 1 (ECL1).

FIGS. 2A-2E. Representation of wild type and mutant CLDN9 protein in HEK293 cells. FIG. 2A and FIG. 2C. Wild type CLDN9 transfected HEK293 cells show the protein is expressed in both the plasma membrane and protoplasm. FIG. 2B and FIG. 2D. Mutant CLDN9 transfected HEK293 cells are showing the mutant protein is limited to the cytosol. GFP-tagged wild type or mutant CLDN9, nucleus and actin (Scale bar: 10 μm). FIG. 2E. Quantification of mutation on subcellular localization of CLDN9 transfected HEK293 cells. The plasma membrane-like organelle and protoplasm were measured for wild type (n=15) and mutant (n=8) CLDN9. Bars represent the mean+SEM of average CLDN9-GFP fluorescence intensity in arbitrary units (AU), *p=6.8×10-6, ns (not significant).

FIGS. 3A-3C. FIG. 3A. Primers used for Sanger sequencing and cloning. FIG. 3B. Homozygous runs (>1Mb) in the proband. All nonsyndromic known deafness genes (AD and AR) in homozygous regions were screened. MET (MIM 164860) gene (NM_001127500; chr7:116,312,459-116,438,440) was located in second homozygous region and 4X coverage is 100% for entire gene. FIG. 3C. Chr: Chromosome, Ref: Reference, Alt: Alternate, GERP: Genomic Evolutionary Rate Profiling, gnomAD: Genome Aggregation Database, dbSNP: The Single Nucleotide Polymorphism database, CADD: Combined Annotation Dependent Depletion.

FIGS. 4A-4B. FIG. 4A. Human CLDN9 DNA. NCBI Reference Sequence: NM_020982.4. FIG. 4B. Human CLDN9 Protein. NCBI Reference Sequence: NP_066192.1.

### DETAILED DESCRIPTION

The disclosure provides a method of treating sensorineural deafness. Sensorineural deafness or hearing loss points at a malfunction of the inner ear or a retrocochlear condition that affects the cochleovestibular nerve within the internal acoustic meatus and cerebellopontine angle or that involves the central auditory pathway (Verbist et al., 2012. Insights Imaging 3(2):139-153).

Disclosed herein is the identification of biallelic mutations in the CLDN9 gene (MIM 615799) associated with nonsyndromic sensorineural deafness. CLDN9 encodes claudin-9, a tight junction protein that localizes to the cell membrane. Three cases of nonsyndromic deafness were identified as being homozygous for a frameshift mutation in CLDN9, c.86delT; p.Leu29ArgfsTer4. In vitro studies in HEK293 cells demonstrated that the mutant CLDN9 does not localize to cell membrane as demonstrated for the wild type protein. The findings described herein demonstrate a major role of CLDN9 in human hearing and establish CLDN9 as a deafness gene in humans.

In one aspect, the disclosure provides a method of characterizing sensorineural deafness in a subject. The method comprises detecting a mutation in CLDN9 in the subject, as described further herein.

The disclosure also provides methods of treating sensorineural deafness. The method comprises administering to a subject having a mutation in the CLDN9 gene a composition that comprises one or more of the following: a polynucleotide that encodes a CLDN9 peptide, a CLDN9 peptide, an agent that blocks expression of a mutant CLDN9 gene, and an agent that corrects the mutation in the CLDN9 gene. The disclosure further provides a method of increasing sensitivity to sound having a frequency of 1000 Hz or greater. The method comprises administering to a subject, such as a subject having a mutation in the CLDN9 gene, a composition that comprises one or more of the following: a polynucleotide that encodes a CLDN9 peptide, a CLDN9 peptide, an agent that blocks expression of a mutant CLDN9 gene, and an agent that corrects the mutation in the CLDN9 gene. In various embodiments, the method comprises administering a combination of the foregoing agents, in a single composition or in separate compositions (optionally administered at different time points). In various aspects, the method comprises detecting the presence of a mutation in the CLDN9 gene in a sample from the subject. In various embodiments, the CLDN9 mutation is preferably a DNA variant classified as pathogenic or likely pathogenic according to American College of Medical Genetics and Genomics (ACMG) criteria. In various aspects of the disclosure, a method is provided which comprises diagnosing the subject with sensorineural deafness when the presence of the mutation in the CLDN9 gene is detected.

In various embodiments, the method comprises detecting the CLDN9 gene mutation c.86delT; p.Leu29ArgfsTer4 or any protein truncating mutation and/or mutation that leads to a ‘loss of function’ or a hypomorphic function of the protein. In related embodiments, mutations in the CLDN9 gene may be detected using next-generation sequencing such as a gene panel, whole exome sequencing (WES), whole-genome sequencing (WGS), Sanger sequencing, and related DNA sequencing methods, polymerase chain reaction (PCR), real-time PCR (RT-PCR), microarray or Multiplex Ligation-dependent Probe Amplification (MLPA) assays for sequence and copy number variants, DNA restriction enzyme digestion and gel electrophoresis, and other DNA mutation detection methods known in the art. Also mutant CLDN9 may be detected via next-generation sequencing or PCR amplification of CLDN9 mRNA or western blotting of CLDN9 protein.

In various aspects, the CLDN9 mutation is detected by examining proteins using western blotting (immunoblot), High-performance liquid chromatography (HPLC), Liquid chromatography—mass spectrometry (LC/MS), antibody dependent methods such as enzyme-linked immunosorbent assay (ELISA), protein immunoprecipitation. protein immunostaining. protein chip methods or other protein detection methods suitable for mutation detection.

The sample may be any biological sample taken from the subject, including, but not limited to, any tissue, cell, or fluid (e.g., blood) which can be analyzed for a trait of interest, such as the presence or amount of a nucleic acid (e.g., CLDN9 mRNA) or a protein (e.g., CLDN9 protein). In various embodiments, the biological sample is a plasma, saliva, urine, or skin sample.

A “subject” as referred to herein, can be any mammal, such as a human. In various embodiments, the subject is an infant, a child, an adolescent or an adult. In various embodiments, the subject is a human aged 5 years or younger. In other aspects, the subject is 13 years or younger, 18 years or younger, 30 years or younger, or over 30 years old. In various embodiments, the subject exhibits hearing loss or insensitivity to sound between frequency thresholds of 0-500 HZ, 500-1000 HZ, or >1000 HZ. In various aspects, the subject exhibits hearing loss or insensitivity to sound in frequencies above 1000 HZ (i.e. 1000 HZ or greater). In related aspects, the subject exhibits hearing loss or insensitivity to sound in frequencies 1000 HZ, 1100 HZ, 1200 HZ, 1300 HZ, 1400 HZ, 1500 HZ, 1600 HZ, 1700 HZ, 1800 HZ, 1900 HZ, 2000 HZ, 2500 HZ, 3000 HZ, 3500 HZ, 4000 HZ, 4500 HZ, 5000 HZ, 5500 HZ, 6000 HZ, 6500 HZ, 7000 HZ, 7500 HZ, 8000 HZ or greater.

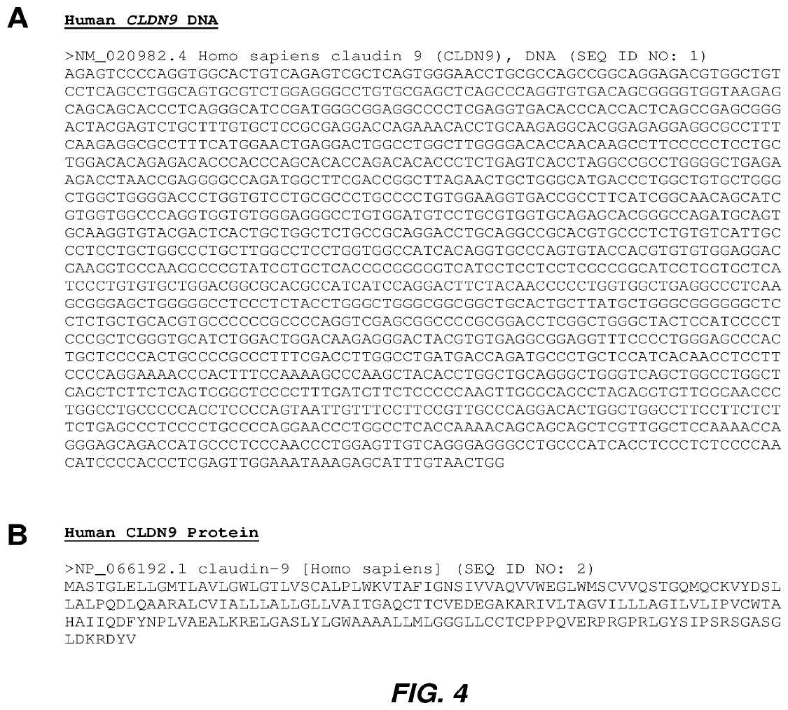

In some embodiments. CLDN9 peptide is administered to the subject. As such, the therapy supplements CLDN9 peptide levels where endogenous, functional CLDN9 levels are inadequate or absent. An exemplary CLDN9 peptide is provided in SEQ ID NO: 2. The disclosure contemplates use of a peptide that comprises at least 80%, at least 85%, at least 90%, at least 95%, or 100% identity to SEQ ID NO: 2. The peptide, in at least one aspect of the disclosure, comprises the peptide sequence shown in FIG. 4B (SEQ ID NO: 2).

In various embodiments, the method comprises administering to the subject a polynucleotide (e.g., a polynucleotide that encodes the CLDN9 peptide/protein, an agent that blocks expression of a mutant CLDN9 gene, and/or an agent that corrects the mutation in CLDN9 gene). Polynucleotides are typically delivered to a host cell via an expression vector, which includes the regulatory sequences necessary for delivery and expression. In some aspects, the constructs described herein include a promoter (e.g., cytomegalovirus (CMV) promoter), a protein coding region (optionally with noncoding (e.g. 3′-UTR) regions that facilitate expression), transcription termination sequences, and/or regulator element sequences. In various aspects, tissue specific or regulatable expression may be desired. In this regard, for example, the Cre-loxP system may be utilized to express a polynucleotide of interest (e.g., CLDN9 gene).

Expression vectors may be viral-based (e.g., retrovirus-, adenovirus-, or adeno-associated virusbased) or non-viral vectors (e.g., plasmids). Non-vector based methods (e.g., using naked DNA, DNA complexes, etc.) also may be employed. Optionally, the vector is a viral vector, such as a lentiviral vector or baculoviral vector, and in various preferred embodiments the vector is an adeno-associated viral vector (AAV). The expression vector may be based on any AAV serotype, including AAV-1, AAV-2, AAV-3, AAV-4, AAV-5. AAV-6, AAV-7, AAV-8, AAV-9, AAV-10, AAV-11, AAV-12, or AAV-13. Polynucleotides also may be delivered via liposomes, nanoparticles, exosomes, microvesicles, hydrodynamic-based gene delivery, or via a “gene-gun.”

In various embodiments, the agent is a polynucleotide that encodes a CLDN9 peptide. The amino acid sequence of CLDN9 is provided as SEQ ID NO: 2 (FIG. 4B). The polynucleotide used in the method optionally encodes the amino acid sequence of SEQ ID NO: 2 or a sequence that is at least 80%, at least 85%, at least 90%, at least 95%, or 100% identical to the amino acid sequence of SEQ ID NO: 2 (which retains the function of CLDN9). Optionally, the polynucleotide comprises SEQ ID NO: 1 (FIG. 4A) or a sequence that is at least 80%, at least 85%, at least 90%, at least 95%, or 100% identical to the polynucleotide sequence of SEQ ID NO: 1 (and which encodes CLDN9). The polynucleotide, in at least one aspect of the disclosure, comprises the polynucleotide sequence shown in FIG. 4A (SEQ ID NO: 1).

In various embodiments, an agent which blocks the expression of a mutant CLDN9 gene is administered, optionally in combination with a polynucleotide that encodes functional CLDN9 or CLDN9, itself. An agent that blocks expression of a mutant CLDN9 gene refers to an agent that interferes with expression of a mutant CLDN9 gene so that mutant CLDN9 gene expression and/or mutant CLDN9 protein levels are reduced compared to basal/ wild-type levels. It will be appreciated that “blocking” expression of a mutant CLDN9 gene does not require 100% abolition of expression and CLDN9 production; any level of reduced expression of aberrant CLDN9 may be beneficial to a subject. Exemplary agents include, but are not limited to, antisense oligonucleotides (ASO), short hairpin RNA (shRNA), small interfering RNA (siRNA), or micro RNA (miRNA). In related aspects, the agent is an antisense oligonucleotide (ASO) used to knock-down (i.e., reduce) the expression of aberrant (i.e. mutant) CLDN9. Suitable CLDN9 ASO sequences are known in the art. It will be appreciated that “blocking” expression of the CLDN9 gene does not require 100% abolition of expression of CLDN9 protein; any level of reduced expression of CLDN9 may be beneficial to a subject. For example, in various aspects, the CLDN9 antisense oligonucleotide reduces the expression of CLDN9 protein by 1, 5, 10, 15, 20, 25, 30, 40, 50, 60, 70, 80 90 or 100%. An ASO is a single-stranded deoxyribonucleotide, which is complementary to an mRNA target sequence. In various aspects, the CLDN9 antisense oligonucleotide targets an exonic or intronic sequence of the CLDN9 gene.

In some embodiments, an agent that corrects the mutation in the CLDN9 gene is employed. In this regard, the agent may comprise components employed in genome-editing techniques, such as designer zinc fingers nucleases (ZFNs), transcription activator-like effectors nucleases (TALENs), or CRISPR-Cas (clustered regularly interspaced short palindromic repeats-CRISPR associated) systems. In some embodiments, the agents are used to modify the sequence of the CLDN9 coding region or a regulatory element and/or non-coding region associated with the CLDN9 gene. In various aspects, genome editing may be used to replace part or all of the CLDN9 gene sequence or alter CLDN9 protein expression levels. An exemplary agent for use in the method of the disclosure is, DNA encoding Cas9 molecules and/or guide RNA (gRNA) molecules. Cas9 and gRNA can be present in a single expression vector or separate expression vectors. Adenoviral delivery of the CRISPR/Cas9 system is described in Holkers et al., Nature Methods (2014), 11(10):1051-1057 which is incorporated by reference in its entirety.

The terms “treating” or “treatment” refer to reducing or ameliorating sensorineural deafness and/or associated disorders and/or symptoms associated therewith. These terms include reducing the severity of the disorder or any symptoms associated therewith. It is appreciated that, although not precluded, “treating” or “treatment” of a disorder or condition does not require that the disorder, condition, or symptoms associated therewith be completely eliminated; any increase in sensitivity to sound or change in a similar sensory perception is contemplated. A change in the ability of a subject to detect sound is readily accomplished through administration of simple hearing tests, such as a tone test commonly administered by an audiologist. In most mammals, a reaction to different frequencies indicates a change in sensory perception. In humans, comprehension of language also is appropriate. A change in perception is indicated by the ability to distinguish different types of acoustic stimuli, such as differentiating language from background noise, and by understanding speech. Speech threshold and discrimination tests are useful for such evaluations. To detect a change in sensory perception (e.g., hearing), a baseline value is recorded prior to treatment using any appropriate sensory test. A subject is reevaluated at an appropriate time period following the method (e.g., 1 day, 3 days, 5 days, 7 days, 14 days, 21 days, 28 days, 2 months, 3 months or more), the results of which are compared to baseline results to determine a change.

A dose of an active agent (e.g., a polynucleotide that encodes a CLDN9 peptide; a CLDN9 peptide; an agent that blocks the expression of a mutant CLDN9 gene; an agent that corrects a mutation in CLDN9 gene) will depend on factors such as route of administration (e.g., local vs. systemic), patient characteristics (e.g., gender, weight, health, side effects), the nature and extent of the sensorineural deafness or associated disorder, and the particular active agent or combination of active agents selected for administration.

Suitable methods of administering a physiologically-acceptable composition, such as a pharmaceutical composition comprising an agent described herein, are well known in the art. In various aspects, more than one route can be used to administer one or more of the agents disclosed herein. A particular route can provide a more immediate and more effective reaction than another route. For example, in certain circumstances, it will be desirable to deliver the composition orally; through injection or infusion by intravenous, intraotic, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; by implantation devices; using nanoparticles; or as a conjugate.

In various aspects, the agent described herein is administered to the inner ear. The most direct routes of administration entail surgical procedures which allow access to the interior of the structures of the inner ear. Inoculation via cochleostomy allows administration of the agent directly to the regions of the inner ear associated with hearing. Cochleostomy involves drilling a hole through the cochlear wall, e.g., in the otic capsule below the stapedial artery as described in Kawamoto et al., Molecular Therapy, 4(6), 575-585 (2001), and release of a pharmaceutical composition. Administration to the endolymphatic compartment is particularly useful for administering an agent to the areas of the inner ear responsible for hearing. Alternatively, the agent can be administered to the semicircular canals via canalostomy. Canalostomy provides for exposure to the vestibular system and the cochlea, whereas cochleostomy does not provide as efficient transduction in the vestibular space. The risk of damage to cochlear function is reduced using canalostomy in as much as direct injection into the cochlear space can result in mechanical damage to hair cells (Kawamoto et al., supra). Administration procedures also can be performed under fluid (e.g., artificial perilymph), which can comprise factors to alleviate side effects of treatment or the administration procedure, such as apoptosis inhibitors or anti-inflammatories.

Another direct route of administration to the inner ear is through the round window, either by injection or topical application to the round window. Administration via the round window is especially preferred for delivering agents to the perilymphatic space.

The agent can be present in or on a device that allows controlled or sustained release, such as an sponge, meshwork, mechanical reservoir or pump, or mechanical implant. For example, a biocompatible sponge or gelfoam soaked in a pharmaceutical composition is placed adjacent to the round window, through which the agent permeates to reach the cochlea. Mini-osmotic pumps provide sustained release of an agent over extended periods of time (e.g., five to seven days), allowing small volumes of composition to be administered, which can prevent mechanical damage to endogenous sensory cells.

A polynucleotide can be introduced ex vivo into cells previously removed from a given subject. Such transduced autologous or homologous host cells can be progenitor cells that are reintroduced into the inner ear of the subject to express, e.g., functional CLDN9. One of ordinary skill in the art will understand that such cells need not be isolated from the patient, but can instead be isolated from another individual and implanted into the patient.

The agent is preferably administered as soon as possible after it has been determined that the subject is at risk for hearing loss (e.g., because of family history or detection of mutant CLDN9 prior to clinical manifestation of hearing impairment) or has demonstrated hearing loss.

It is contemplated the two or more active agents described herein may be administered as part of a therapeutic regimen. Alternatively or in addition, one or more of the active agents may be administered with other therapeutics as part of a therapeutic regimen. The active agent(s) may be administered as a monotherapy or as a combination therapy with other treatments administered simultaneously or metronomically. The term “simultaneous” or “simultaneously” refers to administration of two agents within six hours or less (e.g., within three hours or within one hour each other). In this regard, multiple active (or therapeutic) agents may be administered the same composition or in separate compositions provided within a short period of time (e.g., within 30 minutes). The term “metronomically” means the administration of different agents at different times and at a frequency relative to repeat administration. Active agents need not be administered at the same time or by the same route; preferably, in various embodiments, there is an overlap in the time period during which different active agents are exerting their therapeutic effect.

Additional aspects and details of the disclosure will be apparent from the following examples, which are intended to be illustrative rather than limiting.

### EXAMPLES

### General Methods

### Human Subjects

This study was approved by the Ethics Committee of Ankara University (Turkey) and the Institutional Review Board at the University of Miami (USA). Informed consent was obtained from each participant or, in the case of a minor, from the parents.

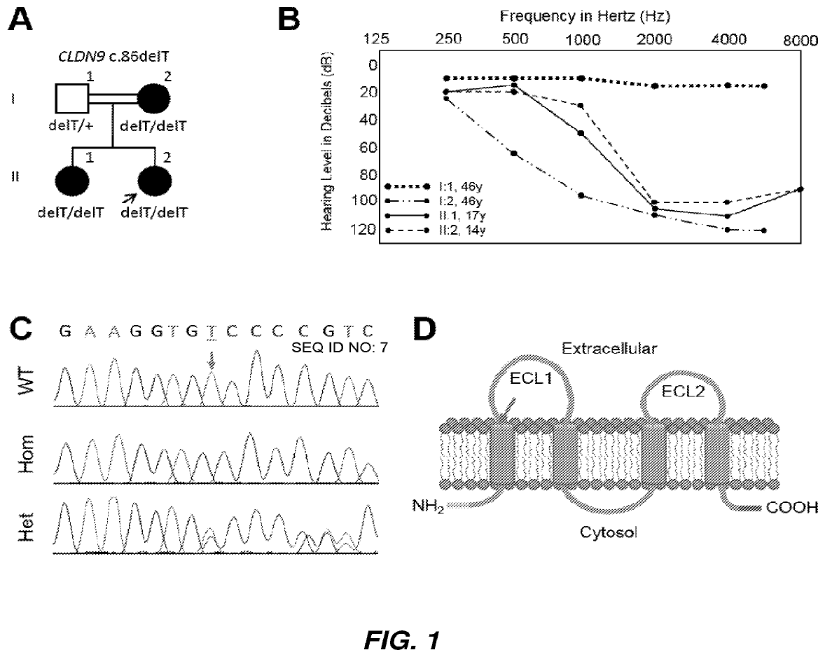

A Turkish family with parental consanguinity was evaluated (FIG. 1A). The diagnosis of sensorineural HL (SNHL) was established following standard audiometric testing in a soundproofed room in accordance with current clinical standards. The pure-tone average over 0.5, 1, 2, and 4 kHz was calculated to determine severity of HL, applied to the better hearing ear: mild=20-40 dB; moderate=41-70 dB; severe=71-95 dB; profound>95 dB (Mazzoli et al. 2003). The clinical evaluation included a thorough physical examination and otoscopy in all cases. DNA was extracted from blood adhering to the standard procedures.

### Sequencing and Bioinformatics

Whole-genome sequencing (WGS) was performed in the proband (II:2) by using the BGISEQ-500 paired-end 100 bp (PE100) (Huang et al. 2017; Bademci et al. 2018). Burrows-Wheeler Aligner was used to align reads to the human reference genome (GRCh37/hg19) (Li and Durbin 2010). Genome Analysis Toolkit was used for variant calling, BreakDancer for detecting structural variants, and CNVnator for copy number variants (McKenna et al. 2010; Chen et al. 2009; Abyzov et al. 2011).

The Genome Aggregation Database (gnomAD), The Single Nucleotide Polymorphism (dbSNP) database, and an internal database was used that contains >8500 exomes from different ethnicities, including >1000 Turkish individuals, and >200 genomes (Karczewski et al. 2019; Sherry et al. 2001). Cutoffs of 0.005 and 0.0005 were used for recessive and dominant variants, respectively, for minor allele frequency thresholds. Filtered variants were used with combination criteria: CADD score >20, GERP score >2, and both PolyPhen-2 and SIFT as “damaging” (Rentzsch et al. 2018; Davydov et al. 2010; Adzhubei et al. 2010; Kumar et al. 2009). Enlis Genome Research software was utilized to identify regions of homozygosity from WGS data (Enlis, Berkeley, Calif.). During the search for novel deafness genes, autozygous regions that are longer than 1 Mb were focused upon. ACMG guidelines and the ClinGen Hearing Loss Gene Curation Expert Panel were followed for interpreting sequence variants (Richards et al. 2015; DiStefano et al. 2019).

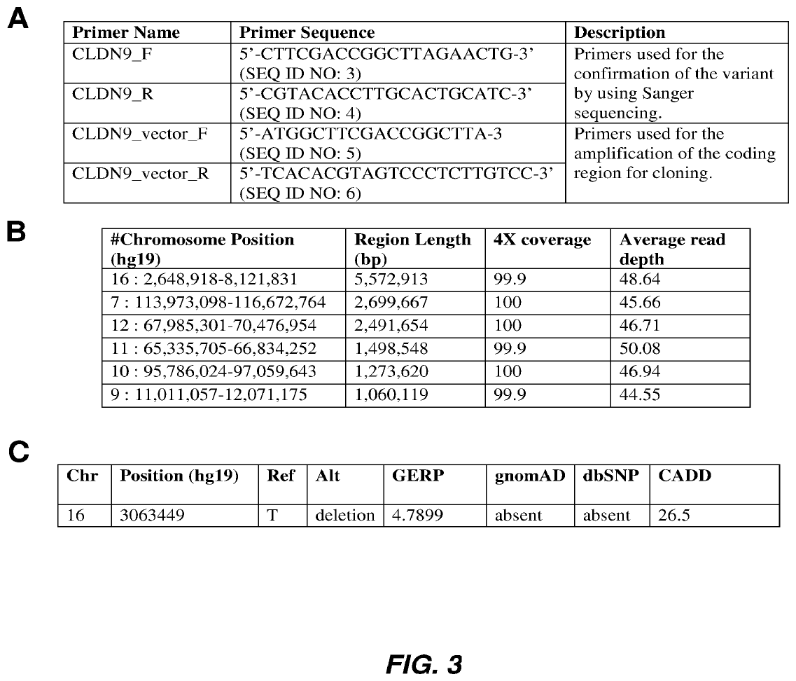

Sanger sequencing was used to confirm and evaluate co-segregation of the candidate variant (FIG. 3A).

In vitro Studies

To assess functionality, CLDN9 was cloned and expressed using a NT-GFP Fusion TOPO TA Expression Kit (cat. no. K4810-01, Invitrogen, Carlsbad, Calif.). Briefly, primers (FIG. 3A) were used to amplify the single coding exon of the gene from the human blood sample CLDN9delT 2398-101 (CLDN9 NM_020982.3:c.86delT) and a wild type control. To isolate the plasmid DNA, a QIAprep Spin Miniprep Kit (cat. no. 27106, Qiagen, Hilden, Germany) was carried out according to manufacturer's instructions. Purified DNA underwent Sanger sequencing again to choose correct orientation of the plasmid and ensure no mutations were introduced.

Wild type and mutant DNA samples were transfected into HEK293 cells using Lipofectamine LTX (cat no. 1533810, Invitrogen, Carlsbad, Calif.). After 48 hours of incubation, cells were fixed in 4% PFA in PBS for 40 minutes at RT, permeabilized in 0.5% Triton X-100 in PBS for 10 min, and co-stained with Hoechst 33342 (cat. no. H3570, Invitrogen, Carlsbad, Calif.) and phalloidin CF633 conjugate (cat. no. 00046, Biotium, Fremont, Calif.). Cells were imaged at 40×magnification with a Zeiss LSM710 confocal microscope (Zeiss, Oberkochen, Germany).

### Statistical Analysis

Two-tailed t-tests were calculated to determine statistical significance by measuring peaks of GFP signal intensity with ZEN software (Zeiss, Oberkochen, Germany). The sample sizes (n), averages, and SEM are listed in each figure legend.

### Example 1

### A Frameshift Variant in CLDN9 is Associated with Nonsyndromic Sensorineural Hearing Loss in a Turkish Family

In a consanguineous family of Turkish origin, three affected individuals had SNHL (FIG. 1A). All three affected members of the family were diagnosed after age 10 years, while age of onset was not clearly delineated. Audiograms showed normal hearing in the father (I:1) and bilateral symmetric profound SNHL in the mother (I:2), moderate SNHL in the proband (II:2), and severe SNHL in an affected elder sister (II:1) (FIG. 1B). Hearing thresholds show a normal hearing level at 500 Hz and a steep decline after 1000 Hz in both sisters. In the 46-year-old mother, both 500 Hz and 1000 Hz hearing levels show significant HL, suggesting that HL has progressed in the mother. Audiograms taken three years later for the sisters (II:1 and 11:2) show the same hearing levels and suggest that there was not a rapid progression in HL. High resolution computed tomography scan of the temporal bone did not show inner ear anomalies in the proband. Gross motor development was normal with no history of balance problems, vertigo, dizziness, or nystagmus. Tandem walking was normal, and the Romberg test was negative. There was no other findings affecting systems other than hearing.

Average read depth for WGS was 47.51× with at least 4× coverage for 98.87% of the genome. After filtering and excluding variants in all known deafness genes, only one variant remained mapping to an autozygous region: CLDN9 NM_020982.3:c.86de1T (p.Leu29ArgfsTer4). The list of autozygous regions is provided in FIG. 3B. The CLDN9 variant is located in a 5.5 Mb autozygous run on chromosome 16, which is the longest of six runs over 1 Mb. The variant was not previously identified in dbSNP or gnomAD and has a CADD score of 26.5 which suggests a deleterious effect. (FIG. 3C).

Sanger sequencing of all four family members showed co-segregation of the variant with the phenotype as an autosomal recessive trait in the family (FIG. 1A and 1C). The variant is located within codon Leu29, which is at the beginning of the first extracellular loop of CLDN9 (FIG. 1D). It was predicted that this mutation causes a frameshift that results in a premature stop codon and a truncated protein. As CLDN9 is a single-exon gene, it is unlikely that a premature stop codon triggers nonsense-mediated decay.

### Example 2

### Detected Variant Impairs CLDN9 Subcellular Localization

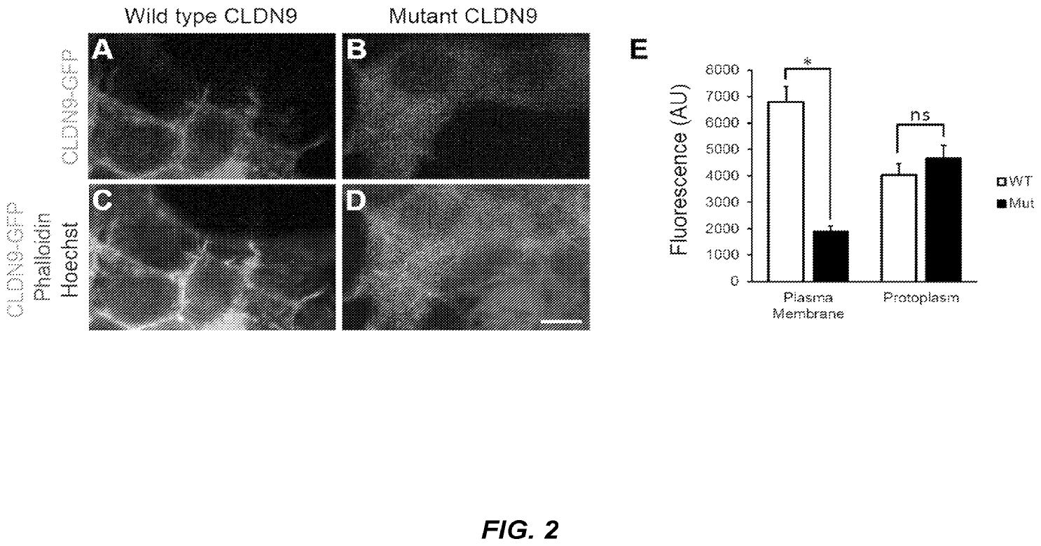

To study the cellular function, a cloning kit was used that included a GFP tag at the N-terminal (Nt). The c.86delT variant occurs downstream of the Nt. It was hypothesized that wild type CLDN9 would be located in the plasma membrane, where it function as integral membrane proteins, and that the mutant CLDN9 would result in a truncated protein. As hypothesized, in the wild type, the GFP is primarily localized in the plasma membrane (FIG. 2A). Strikingly, the GFP is localized to the cytosol in the mutant protein (FIG. 2B). Moreover, the GFP signal is decreased in the mutant compared to the wild type.

The intensity of the GFP signal in the plasma membrane-like structure and protoplasm was measured for both the CLDN9 control (FIG. 2C) and mutant (FIG. 2D). The CLDN9 mutant had a decreased signal in the plasma membrane compared to the wild type (FIG. 2E). Instead, the CLDN9 is primarily found within the cytosol. However, there was no difference in intensity within the protoplasm between the two groups. This suggests that the variant disrupts the migration of CLDN9 to the plasma membrane.

Disclosed herein, CLDN9 is a novel gene for autosomal recessive nonsyndromic HL. Evidence comes from a consanguineous family with a truncating mutation. To support this conclusion, in vitro studies described herein show that the truncated CLDN9 is confined to the cytosol whereas in the wild type, it is located in the plasma membrane. CLDN9 is a membrane protein and thus cannot function properly within the cytosol. This highlights that the c.86delT variant disrupts the function of CLDN9 and results in SNHL as a consequence.

Nakano et al. (2009) previously reported a claudin-9-deficient mouse strain, nfm329 which was generated by using ENU-induced mutagenesis. After evaluating startle responses and measuring auditory brainstem responses, it was concluded that nmf329/nmf329 mice had severe HL at P16, which is indicative of early onset, given that the onset of hearing in wild type mice is at P15. Wild type and nmf329/+mice had no difference in hearing thresholds. Similarly, the heterozygous father in the study has normal hearing. No other abnormalities were detected in the nmf329/nmf329 mice, following balance tests and histopathological experiments. Likewise in the family, the affected individuals had no additional clinical findings in the physical examination. This draws parallels between the CLDN9 homozygous individuals and the nmf329/nmf329 mice, in which genetic variations both result in recessively inherited nonsyndromic HL.

Audiograms obtained from the family have an unusual configuration. Low frequencies remain normal, while frequencies over 1000 Hz steeply decline. Studies on the nmf329 mouse line conducted by Nakano et al. (2009) further corroborate the findings. In the nmf329/nmf329 cochlea at P28, the organ of Corti was collapsed at the basal turn, lacking paracellular spaces and one of the three rows of outer hair cells (OHCs). However, the organ of Corti appeared intact at the apical turn with three rows of OHCs in nmf329 homozygotes. Similar to the results of the ABR experiment, wild type and nmf329/+ mice were morphologically normal along the entire length of the cochlea. To better understand the progression of hair cell degeneration, Nakano et al. performed histological experiments at different stages of development. In nmf329/nmf329 mice at P8, both inner hair cells (IHCs) and OHCs were present and intact, but most of the OHCs at the basal turn had degenerated by P14. Interestingly, the effect was less pronounced at the cochlear apex. It is important to note the missing paracellular spaces in nmf329 homozygotes. Since CLDN9 is involved in paracellular permeability, this deformity is likely to impair functionality. Cochleae taken from nmf329/nmf329 mice at P80 show that the rapid degeneration that occurred in early development abated with a few OHCs remaining at the basal turn. This slow progression of HL is also observed in the family; the proband (younger sister) has moderate HL, the older sister has severe HL, and the mother is profoundly deaf. It suggests that the degeneration of hair cells is accelerated during adolescence and into adulthood, then decelerates later in life. It is concluded that CLDN9 is essential for the hair cells in the base of cochlea from early on and that c.86delT variant results in steeply sloping high frequency moderate to profound SNHL.

The findings presented herein are clinically significant because the importance of CLDN9 in human hearing was unexplored. Until this study. CLND9 was not known to be associated with HL in humans. Identifying this variant is one step closer to mapping the complete genetic landscape of deafness in humans.

### REFERENCES

Abyzov A et al.,(2011) CNVnator: an approach to discover, genotype, and characterize typical and atypical CNVs from family and population genome sequencing. Genome Res 21:974-984.

Adzhubei I A et al., (2010) A method and server for predicting damaging missense mutations. Nat Methods 7:248-249.

Bademci G et al., (2018) MPZL2 is a novel gene associated with autosomal recessive nonsyndromic moderate hearing loss. Hum Genet 137:479-486.

Chen K et al., (2009) BreakDancer: an algorithm for high-resolution mapping of genomic structural variation. Nat Methods 6:677-681.

Davydov E V et al., (2010) Identifying a high fraction of the human genome to be under selective constraint using GERP++. PLoS Comput Biol. 6:e1001025.

DiStefano M T et al., ClinGen Hearing Loss Clinical Domain Working Group (2019) ClinGen expert clinical validity curation of 164 hearing loss gene-disease pairs. Genet Med.

Exome Variant Server, NHLBI GO Exome Sequencing Project (ESP), Seattle, Wash. Accessed 2 April 2019.

Huang J et al., (2017) A reference human genome dataset of the BGISEQ-500 sequencer. GigaScience 6:1-9.

Karczewski K J et al., (2019) Variation across 141,456 human exomes and genomes reveals the spectrum of loss-of-function intolerance across human protein-coding genes. bioRxiv 531210.

Kumar P et al., (2009) Predicting the effects of coding non-synonymous variants on protein function using the SIFT algorithm. Nat Protoc 4:1073-1081.

Li H, Durbin R (2010) Fast and accurate long-read alignment with Burrows-Wheeler Transform. Bioinformatics 26:589-595.

Nakano Y et al., (2009) A claudin-9-based ion permeability barrier is essential for hearing. PLoS Genet 5:e1000610.

Nance W E (2003) The genetics of deafness. Ment Retard Dev Disabil Res Rev 9:109-119.

Mazzoli M et al., (2003) Recommendations for the description of genetic and audiological data for families with nonsyndromic hereditary hearing impairment. Audiol Med 1:148-150.

McKenna A et al., (2010) The Genome Analysis Toolkit: a MapReduce framework for analyzing next-generation DNA sequencing data. Genome Res 20:1297-1303.

Mehl A L, Thomson V (2002) The Colorado newborn hearing screening project, 1992-1999: on the threshold of effective population-based universal newborn hearing screening. Pediatrics 109:e7.

Morton C C, Nance WE (2006) Newborn hearing screening—a silent revolution. N Engl J Med 354:2151-2164.

Rentzsch P et al., (2018) CADD: predicting the deleteriousness of variants throughout the human genome. Nucleic Acids Res. 47:D886-D894.

Richards S et al., ACMG Laboratory Quality Assurance Committee (2015) Standards and guidelines for the interpretation of sequence variants: a joint consensus recommendation of the American College of Medical Genetics and Genomics and the Association for Molecular Pathology. Genet Med 17:405-424.

Shearer A E, Smith R J (2015) Massively Parallel Sequencing for Genetic Diagnosis of Hearing Loss: The New Standard of Care.Otolaryngol Head Neck Surg 153:175-82.

Sherry S T, et al., (2001) dbSNP: the NCBI database of genetic variation. Nucleic Acids Res 29:308-311.

Van Camp G, Smith RJH. Hereditary Hearing Loss Homepage. Accessed 31 Mar. 2019.

Van Itallie C M, Anderson J M (2004) The molecular physiology of tight junction pores. Physiology (Bethesda) 19:331-338.

Wilcox E R, et al., (2001) Mutations in the gene encoding tight junction claudin-14 cause autosomal recessive deafness DFNB29. Cell 104:165-172.

What is claimed:

1. A method of treating sensorineural deafness in a human subject in need thereof, the method comprising:
administering to the subject having a mutation in a claudin-9 gene (CLDN9) a composition that comprises a polynucleotide that encodes a CLDN9 peptide; a CLDN9 peptide; an agent that blocks the expression of a mutant CLDN9 gene; an agent that corrects a mutation in CLDN9 gene, or a combination of any of the foregoing.

2. The method of claim 1, wherein the mutation in the CLDN9 gene is c.86delT;
p.Leu29ArgfsTer4.

3. The method of claim 1, wherein the agent is a CLDN9 peptide.

4. The method of claim 1, wherein the agent is a polynucleotide encoding the CLDN9 peptide.

5. The method of claim 1, wherein the agent that blocks the expression of a mutant CLDN9 gene is an CLDN9 antisense oligonucleotide or CRISPR Cas9 protein and one or more guide RNA molecules, TALEN or zinc finger nuclease (ZFN).

6. The method of claim 1, wherein the agent that corrects the mutation in CLDN9 gene is a CRISPR Cas9 protein and one or more guide RNA molecules, TALEN or zinc finger nuclease (ZFN).

7. The method of any one of claims 1-6, wherein the method comprises, prior to the administration step, detecting the presence of a mutation in the claudin-9 (CLDN9) gene in a sample from the subject.

8. The method of claim 7, wherein the mutation in the CLDN9 gene is c.86delT;
p.Leu29ArgfsTer4.

9. The method of any one of claims 1-8, wherein the subject exhibits reduced sensitivity to sound in frequencies above 1000 HZ.

10. A method of characterizing hearing loss in a human subject, the method comprising detecting a c.86delT; p.Leu29ArgfsTer4 mutation in the claudin-9 (CLDN9) gene in a sample from the subject.

11. A method of increasing a subject's sensitivity to sound in frequencies above 1000 HZ, the method comprising administering to a subject in need thereof a composition that comprises a polynucleotide that encodes a CLDN9 peptide; a CLDN9 peptide; an agent that blocks the expression of a mutant CLDN9 gene; an agent that corrects a mutation in CLDN9 gene, or a combination of any of the foregoing.

12. The method of claim 11, wherein the mutation in the CLDN9 gene is c.86delT;
p.Leu29ArgfsTer4.

13. The method of claim 11, wherein the agent is a CLDN9 peptide.

14. The method of claim 11, wherein the agent is a polynucleotide encoding the CLDN9 peptide.

15. The method of claim 11, wherein the agent that blocks the expression of a mutant CLDN9 gene is an CLDN9 antisense oligonucleotide or CRISPR Cas9 protein and one or more guide RNA molecules, TALEN or zinc finger nuclease (ZFN).

16. The method of claim 11, wherein the agent that corrects the mutation in CLDN9 gene is a CRISPR Cas9 protein and one or more guide RNA molecules, TALEN or zinc finger nuclease (ZFN).

17. The method of any one of claims 11-16, wherein the method comprises, prior to the administration step, detecting the presence of a mutation in the claudin-9 (CLDN9) gene in a sample from the subject.

18. The method of claim 17, wherein the mutation in the CLDN9 gene is c.86delT; p.Leu29ArgfsTer4.


SEQUENCE LISTING (7 sequences)

SEQ ID NO: 1 - DNA, 1583 bp, Homo sapiens
agagtccccaggtggcactgtcagagtcgctcagtgggaacctgcgccagccggcaggagacgtggctgtcctcagcctggcagtgcgtctggagggcctgtgcgagctcagcccaggtgtgacagcggggtggtaagagcagcagcaccctcagggcatccgatgggcggaggcccctcgaggtgacacccaccactcagccgagcgggactacgagtctgctttgtgctccgcgaggaccagaaacacctgcaagaggcacggagaggaggcgcctttcaagaggcgcctttcatggaactgaggactggcctggcttggggacaccaacaagccttccccctcctgctggacacagagacacccacccagcacaccagacacaccctctgagtcacctaggccgcctggggctgagaagacctaaccgaggggccagatggcttcgaccggcttagaactgctgggcatgaccctggctgtgctgggctggctggggaccctggtgtcctgcgccctgcccctgtggaaggtgaccgccttcatcggcaacagcatcgtggtggcccaggtggtgtgggagggcctgtggatgtcctgcgtggtgcagagcacgggccagatgcagtgcaaggtgtacgactcactgctggctctgccgcaggacctgcaggccgcacgtgccctctgtgtcattgccctcctgctggccctgcttggcctcctggtggccatcacaggtgcccagtgtaccacgtgtgtggaggacgaaggtgccaaggcccgtatcgtgctcaccgcgggggtcatcctcctcctcgccggcatcctggtgctcatccctgtgtgctggacggcgcacgccatcatccaggacttctacaaccccctggtggctgaggccctcaagcgggagctgggggcctccctctacctgggctgggcggcggctgcactgcttatgctgggcggggggctcctctgctgcacgtgccccccgccccaggtcgagcggccccgcggacctcggctgggctactccatcccctcccgctcgggtgcatctggactggacaagagggactacgtgtgaggcggaggtttcccctgggagcccactgctccccactgccccgccctttcgaccttggcctgatgaccagatgccctgctccatcacaacctccttccccaggaaaacccactttccaaaagcccaagctacacctggctgcagggctgggtcagctggcctggctgagctcttctcagtggggtcccctttgatgttctcccccaagttgggcagcctagaggtgttgggaaccctggcctgcccccacctccccagtaattgtttccttccgttgcccaggacactggctggccttccttctcttctgagccctcccctgccccaggaaccctggcctcaccaaaacagcagcagctcgttggctccaaaaccagggagcagaccatgccctcccaaccctggagttgtcagggagggcctgcccatcacctccctctccccaacatccccaccctcgagttggaaataaagagcatttgtaactgg

SEQ ID NO: 2 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 3 - DNA, 20 bp, Artificial Sequence
  Synthetic Polynucleotide
cttcgaccggcttagaactg

SEQ ID NO: 4 - DNA, 20 bp, Artificial Sequence
  Synthetic Polynucleotide
cgtacaccttgcactgcatc

SEQ ID NO: 5 - DNA, 18 bp, Artificial Sequence
  Synthetic Polynucleotide
atggcttcgaccggctta

SEQ ID NO: 6 - DNA, 22 bp, Artificial Sequence
  Synthetic Polynucleotide
tcacacgtagtccctcttgtcc

SEQ ID NO: 7 - DNA, 15 bp, Artificial Sequence
  Synthetic Polynucleotide
gaaggtgtccccgtc